(19) United States
(12) Patent Application Publication
(10) Pub. No.: US 2017/0227518 A1
(43) Pub. Date: Aug. 10, 2017

(54) SAMPLE DETECTING DEVICE

(71) Applicant: Aidmics Biotechnology Co., Ltd., Taipei City (TW)

(72) Inventors: Cheng-Ming LIN, Taipei City (TW); Chang-Yu CHEN, Taipei City (TW); Tsun-Chao CHIANG, Taipei City (TW); Shu-Sheng LIN, Taipei City (TW)

(21) Appl. No.: 15/411,294

(22) Filed: Jan. 20, 2017

(30) Foreign Application Priority Data
Feb. 5, 2016 (TW) 105104132

(51) Int. Cl. G01N 33/487; H04M 1/725; G01N 15/14; G02B 21/36; G02B 21/06

(52) U.S. Cl. CPC G01N 33/487; G02B 21/36; G02B 21/06; G01N 15/1456; H04M 1/72527; G01N 2015/0065

(57) ABSTRACT

A sample detecting device is used in cooperation with an image capturing device. The sample detecting device includes a first assembly and a second assembly. The first assembly includes a light emitting unit and a light-permeable unit. The light-permeable unit is disposed at one side of the light emitting unit. The first assembly and the second assembly match and connect with each other to form a sample containing space. The second assembly includes a body and a convex lens. The body has a first cavity portion and a second cavity portion. The light-permeable unit is disposed in the first cavity portion, and the convex lens is disposed in the second cavity portion. The light emitted from the light emitting unit sequentially passes through the light-permeable unit and the convex lens and leaves the body.

### CROSS REFERENCE TO RELATED APPLICATIONS

This Non-provisional application claims priority under 35 U.S.C. §119(a) on Patent Application No(s). 105104132 filed in Taiwan, Republic of China on Feb. 5, 2016, the entire contents of which are hereby incorporated by reference.

### BACKGROUND OF THE INVENTION

Field of Invention

This invention relates to a sample detecting device.

Related Art

Bearing sons and daughters is responsibility of both parents. Although the whole pregnancy is in progress in the female body, the fertility of the male spermatozoon plays a decisive role. According to research statistics, male sterility causes three infecund couples among ten infecund couples. In general, for detecting the male sterility, semen needs to be injected into a transparent plastic box and then carried to the reproduction medicine center in two hours with a warmth keeping method by hands for the detection of semen volume, pH value, spermatozoon (amount, vitality, form, inflammation or not) and antibody test of spermatozoon. The above process may bring some kind of psychological disorder for some males, so that they don't want to receive the detection. Besides, specially going to the reproduction medicine center for the detection also will cause much inconvenience. For example, they need to take some time off according to the working hours of the reproduction medicine center.

Since the technology is improved a lot, the existing mobile communication devices, such as cell phones or tablet computers, not only have good portability but also are equipped with a certain level of computing capability so that they can process some easy computation which only could be performed by the computer of a laboratory before. Therefore, there is a need to present a kind of detecting apparatus, which has a lower price and can be used in cooperation with a cell phone or a tablet computer, for the male sterility, so that the males can do a simple detection by themselves at home. Thus, it can be made sure that the freshest sample is detected, and the embarrassment caused by going to the reproduction medicine center for the detection can be avoided. Besides, the economic burden can be reduced, the detection process can be simplified, and the detection result can be obtained without waiting for several days.

### SUMMARY OF THE INVENTION

In view of the foregoing subject, an objective of this invention is to provide a sample detecting device which can be used in cooperation with a mobile communication device, wherein a first assembly and a second assembly can be easily assembled to form a sample containing space for the detection.

In order to achieve the above objective, the present invention discloses a sample detecting device, which is used in cooperation with an image capturing device and includes a first assembly and a second assembly. The first assembly includes a light emitting unit and a light-permeable unit. The light-permeable unit is disposed at one side of the light emitting unit. The first assembly and the second assembly match and connect with each other to form a sample containing space. The second assembly includes a body and a convex lens. The body has a first cavity portion and a second cavity portion. The light-permeable unit is disposed in the first cavity portion, and the convex lens is disposed in the second cavity portion. The light emitted from the light emitting unit sequentially passes through the light-permeable unit and the convex lens and leaves the body.

In one embodiment, the light emitting unit includes a light source and a light output hole. The light source is disposed in the light emitting unit, and at least a part of the light emitted by the light source is outputted through the light output hole.

In one embodiment, the light emitting unit includes a light source and a housing. The light source is disposed on a side of the housing, and the light-permeable unit is disposed on the light source.

In one embodiment, the first cavity portion communicates with the second cavity portion.

In one embodiment, the first cavity portion doesn't communicate with the second cavity portion.

In one embodiment, the light-permeable unit includes a base portion and a sample getting portion. The base portion includes a first connection end and a second connection end opposite to the first connection end. The light-permeable unit is connected with the light emitting unit through the first connection end. The sample getting portion is disposed at the second connection end of the base portion.

In one embodiment, the light-permeable unit includes a base portion and a sample getting portion. The base portion includes a first connection end and a second connection end opposite to the first connection end. The light-permeable unit is connected with the light emitting unit through the first connection end. The sample getting portion is disposed at the second connection end of the base portion. The sample getting portion includes a soft light-permeable sub-portion, a hard light-permeable sub-portion and a microstructure sub-portion. The soft light-permeable sub-portion is connected with the second connection end of the base portion. The hard light-permeable sub-portion has a sample getting surface, and the microstructure sub-portion is disposed on the sample getting surface.

In one embodiment, the material of the hard light-permeable sub-portion includes glass, quartz, PMMA (Polymethylmethacrylate) or PC (Polycarbonate).

In one embodiment, the material of the soft light-permeable sub-portion includes silicone.

In one embodiment, the microstructure sub-portion is a bar-like or granular structure.

In one embodiment, the light-permeable unit includes a base portion and a sample getting portion. The base portion includes a first connection end and a second connection end opposite to the first connection end. The light-permeable unit is connected with the light emitting unit through the first connection end. The sample getting portion includes a third cavity portion disposed opposite to the first connection end of the base portion.

In one embodiment, the image capturing device is a cell phone, a tablet computer, a camera, a network camera or a driving recorder.

As mentioned above, the sample detecting device of this invention can be used in cooperation with the mobile communication device. Besides, the first assembly and the second assembly can be easily assembled to form the sample containing space, so that the sample can be contained in the sample containing space and imaged. The captured microscopic image can be transmitted to the mobile communication device for the computation and thus the detection. Therefore, the threshold of the biological sample detection can be lowered down a significantly, so that the general user also can rapidly perform a simple biological sample detection at home.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a schematic appearance diagram of the assembled sample detecting device of an embodiment of the invention;

FIG. 1B is a schematic sectional exploded diagram of the sample detecting device of FIG. 1A taken along the line A-A;

FIG. 1C is a schematic sectional assembled diagram of the sample detecting device of FIG. 1A taken along the line A-A;

FIG. 1D is a schematic diagram of the sample detecting of an embodiment of the invention in cooperation with an external image capturing device;

FIG. 2A is a schematic sectional exploded diagram of the sample detecting device of another embodiment of the invention taken along the line A-A;

FIG. 2B is a schematic sectional assembled diagram of the sample detecting device of another embodiment of the invention taken along the line A-A;

FIG. 3A is a schematic sectional exploded diagram of the sample detecting device of another embodiment of the invention taken along the line A-A;

FIG. 3B is a schematic sectional assembled diagram of the sample detecting device of another embodiment of the invention taken along the line A-A;

FIG. 4A is a schematic sectional diagram of the sample detecting device of another embodiment of the invention taken along the line A-A;

FIG. 4B is a schematic sectional assembled diagram of the sample detecting device of another embodiment of the invention taken along the line A-A;

FIG. 5A is a schematic side-view of the light-permeable unit of the sample detecting device of an embodiment of the invention; and

FIG. 5B is a schematic side-view of the light-permeable unit of the sample detecting device of another embodiment of the invention.

### DETAILED DESCRIPTION OF THE INVENTION

The present invention will be apparent from the following detailed description, which proceeds with reference to the accompanying drawings, wherein the same references relate to the same elements.

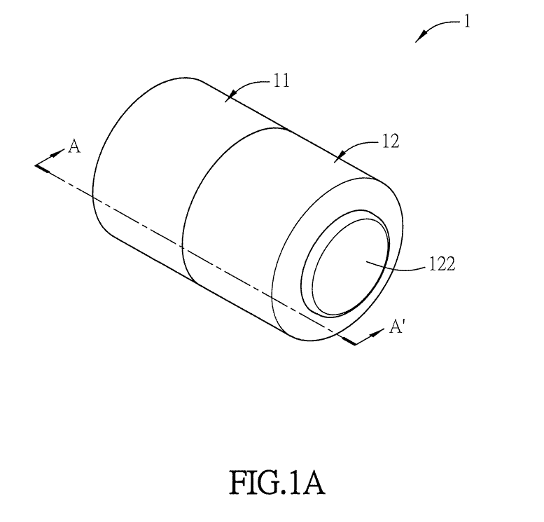

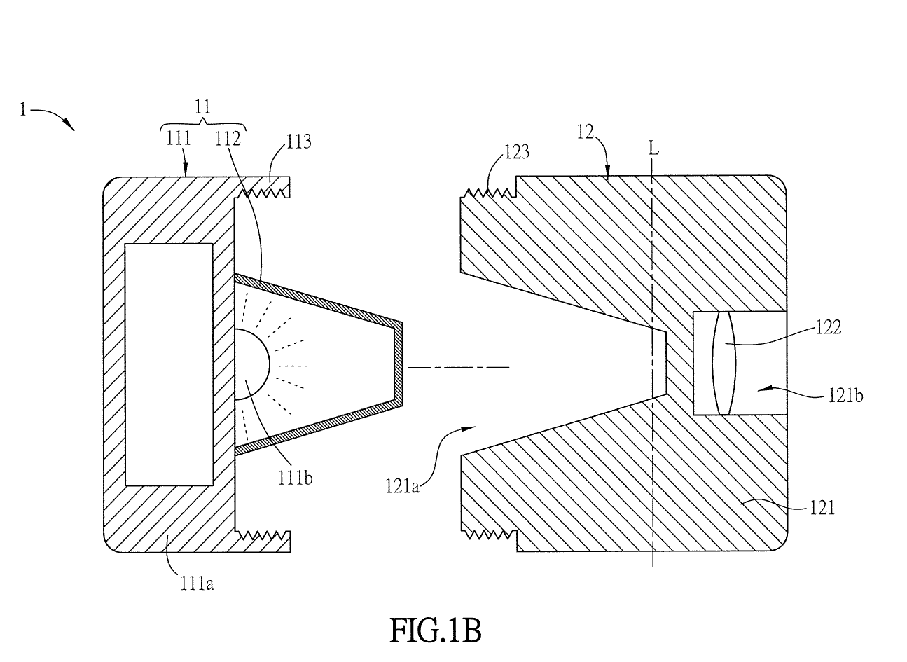

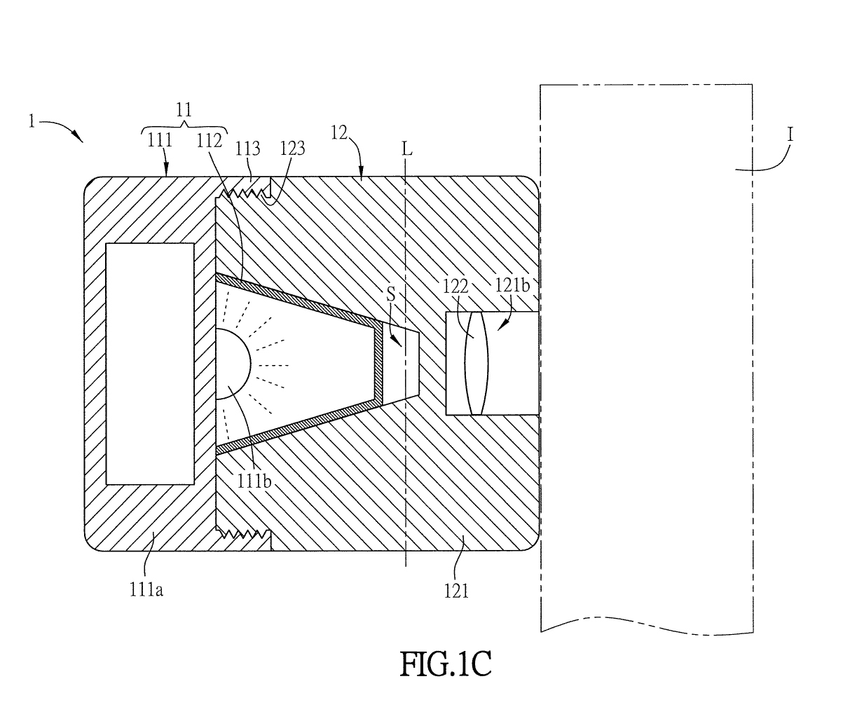

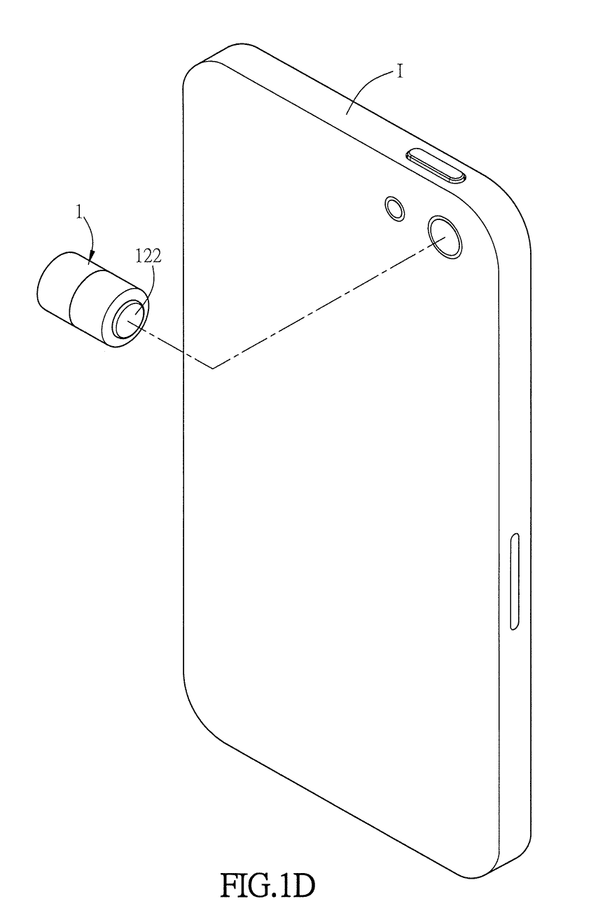

FIG. 1A is a schematic appearance diagram of the assembled sample detecting device of an embodiment of the invention. FIG. 1B is a schematic sectional exploded diagram of the sample detecting device of FIG. 1A taken along the line A-A. FIG. 1C is a schematic sectional assembled diagram of the sample detecting device of FIG. 1A taken along the line A-A. FIG. 1D is a schematic diagram of the sample detecting of an embodiment of the invention in cooperation with an external image capturing device. As shown in FIG. 1D, the sample detecting device 1 of this embodiment is used in cooperation with an external image capturing device I.

As shown in FIGS. 1A and 1B, the sample detecting device 1 includes a first assembly 11 and a second assembly 12. The assembly refers to an object which is composed of a plurality of units, modules or elements to have a part of the function. The first assembly 11 includes a light emitting unit 111 and a light-permeable unit 112. The light emitting unit 111 includes a housing 111a and a light source 111b. The light source 111b is disposed on a side of the housing 111a, and the light-permeable unit 112 is disposed above the light source 111b. The light-permeable unit 112 covers the light source 111b in this embodiment, but this invention is not limited thereto, as long as the light-permeable unit 112 and the light source 111b are disposed on the same side of the housing 111a and at least a part of the light emitted by the light source 111b can be outputted through the light-permeable unit 112. The light-permeable unit 112 can be a light guiding rod or a unit which can help the even diffusion of the light. The second assembly 12 includes a body 121 and a convex lens 122. In this embodiment, the body 121 includes a first cavity portion 121a and a second cavity portion 121b, and the first cavity portion 121a doesn't communicate with the second cavity portion 121b. The convex lens 122 is disposed in the second cavity portion 121b. To be noted, the junction between the first cavity portion 121a and the second cavity portion 121b is permeable to light, so that the light emitted by the light emitting unit 111 can sequentially pass through the light-permeable unit 112 and the convex lens 122 and then leave the body 121.

As shown in FIGS. 1B and 1D, when the sample detecting device 1 is used in cooperation with the image capturing device, the camera lens of the image capturing device I is disposed corresponding to the outlet of the second cavity portion 121b, i.e. the position where the light is emitted out of the sample detecting device 1, to receive the light emitted from the sample detecting device 1. Then, the received light can be captured into a digital image signal, which will be transmitted to an external electronic device or a cloud server through a communication module for the retouch, analysis, storage or development. The external electronic device can be a cell phone, a tablet computer, a notebook computer or a desktop computer. The cloud server refers to the server which can provide cloud computation or storage service. The communication module can be a wireless communication module (ex. WiFi or Bluetooth) or a wired communication module (a transmission cable). In the embodiment of FIG. 1D, the image capturing device I and the external electronic device are the same device.

As shown in FIGS. 1B and 1C, the first assembly 11 and the second assembly 12 can match and connect each other. When the first assembly 11 and the second assembly 12 are connected to each other, the light-permeable unit 112 is accommodated in the first cavity portion 121a and the first assembly 11 and the second assembly 12 collectively form a sample containing space S. The sample containing space S can contain the sample. In this invention, the sample can be a liquid sample or a solid sample, such as semen or blood, but this invention is not limited thereto. According to different requirements, the sample may be a biological sample such as tissue section, cell culture liquid or tissue fluid, or a non-biological sample such as mineral, leather or high polymer. In this embodiment, the first assembly 11 can further include a first connecting portion 113 disposed on the housing 111a, and the body 121 can further include a second connecting portion 123 disposed on the body 121. The first connecting portion 113 and the second connecting portion 123 are disposed correspondingly and connected to each other, so that the first assembly 11 and the second assembly 12 are fixed and connected to each other relatively to form the sample containing space S. In this embodiment, the first assembly 11 includes the first connecting portion 113 and the second assembly 12 includes the second connecting portion 123. The first connecting portion 113 and the second connecting portion 123 are a pair of matching screw structures which can screw each other. To be noted, the above embodiment is just for the illustration but not for limiting the scope of this invention. As long as the first assembly 11 and the second assembly 12 can be relatively fixed and connected to each other to form the sample containing space S, other technical means can be used to achieve their connection, such as tenon, locking, magnetic attraction or adhesion. When the first assembly 11 and the second assembly 12 are connected to each other, the light emitting unit 111 provides a backlight source for the sample (not shown). The light emitted by the light emitting unit 111 passes through the light-permeable unit 112 and then diffuses in the sample and passes through the sample. Then, the light passes through the convex lens 122 and enters the image capturing device I for the imaging. The image capturing device I will capture the image formed by the convex lens 122 into a digital picture signal.

To be noted, in the above embodiment, when the sample (not shown) is accommodated in the sample containing space S, at least a part of the sample is disposed on the focus plane L of the convex lens 122, so that the light diffusing in the sample and passing through the sample can be imaged to the image capturing device I through the convex lens 122. However, due to the digital/optical zoom function of the image capturing device I, the focus plane L of the convex lens 122 can move in a region instead of being a fixed plane. Moreover, for the convenient use, in a design variation, the light emitting unit 111 can further include a battery (not shown) disposed in the housing 111a for providing power for the light source 111b. In another design variation, the light emitting unit 111 can further include a switch (not shown) disposed in the housing 111a and electrically connected between the battery (not shown) and the light source 111b for controlling the power that the battery provides for the light source 111b.

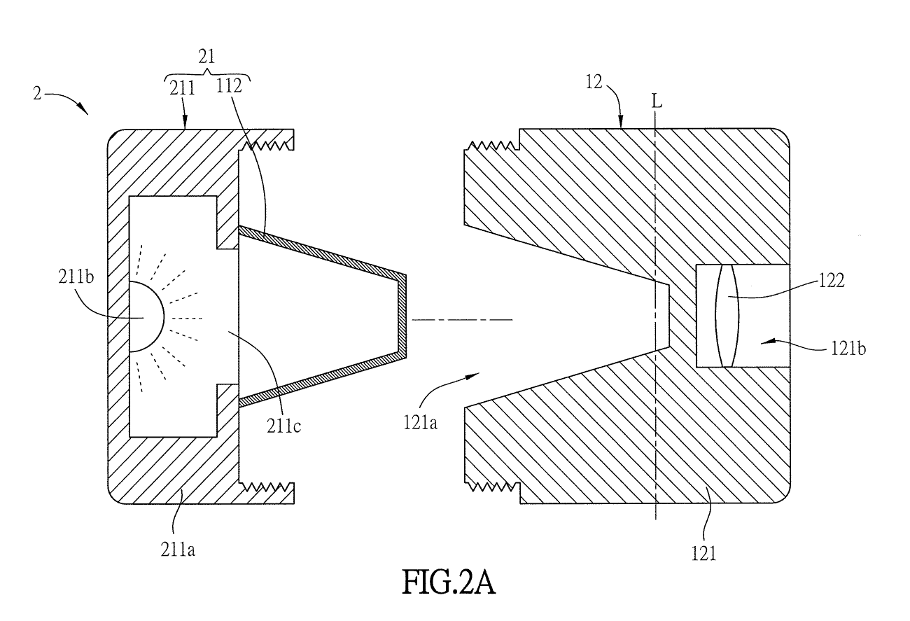

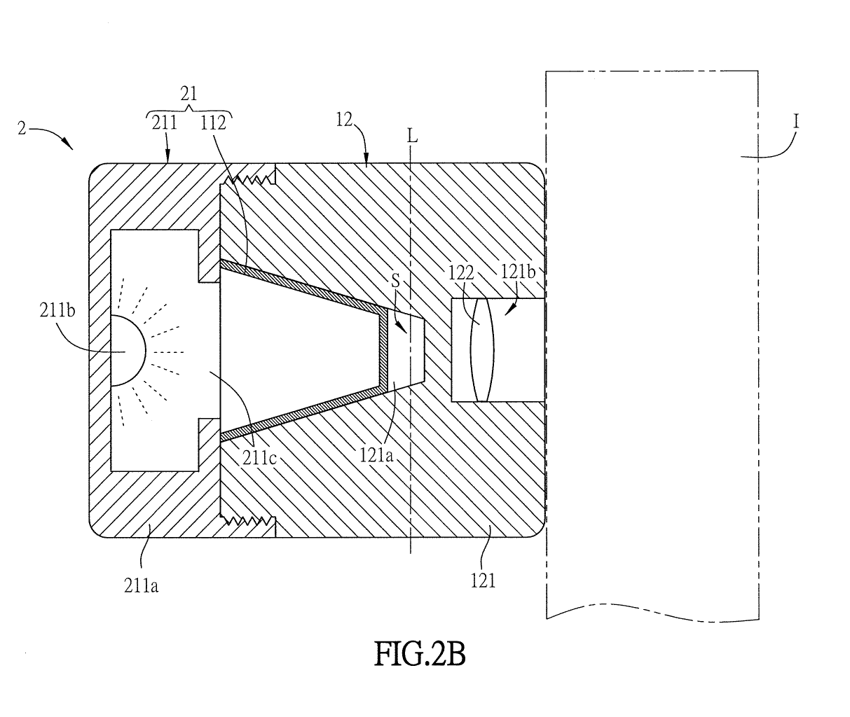

FIG. 2A is a schematic sectional exploded diagram of the sample detecting device of another embodiment of the invention taken along the line A-A, and FIG. 2B is a schematic sectional assembled diagram of the sample detecting device of another embodiment of the invention taken along the line A-A. As shown in FIGS. 2A and 2B, the composition of the sample detecting device 2 is approximately similar to the sample detecting device 1. The main difference between them is that the light emitting unit 211 of the first assembly 21 of the sample detecting device 2 includes a housing 211a, a light source 211b and a light output hole 211c. The light source 211b is disposed within the housing 211a, at least a part of the light emitted by the light source 211b is outputted through the light output hole 211c, and the light-permeable unit 112 is disposed on the light output hole 211c. In this embodiment, the light-permeable unit 112 covers the light output hole 211c, but this invention is not limited thereto, as long as the light-permeable unit 112 and the light output hole 211c are disposed on the same side of the housing 211a and over the light output hole 211c.

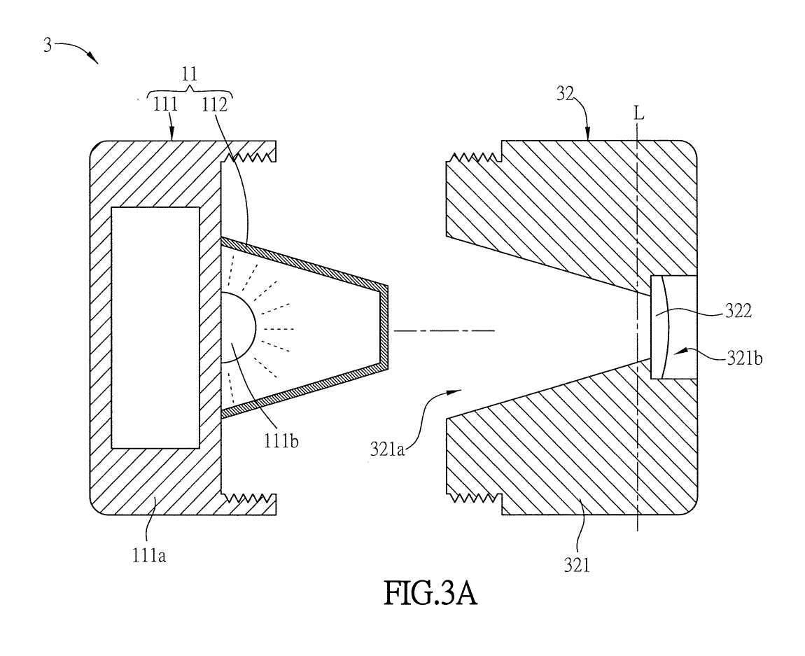

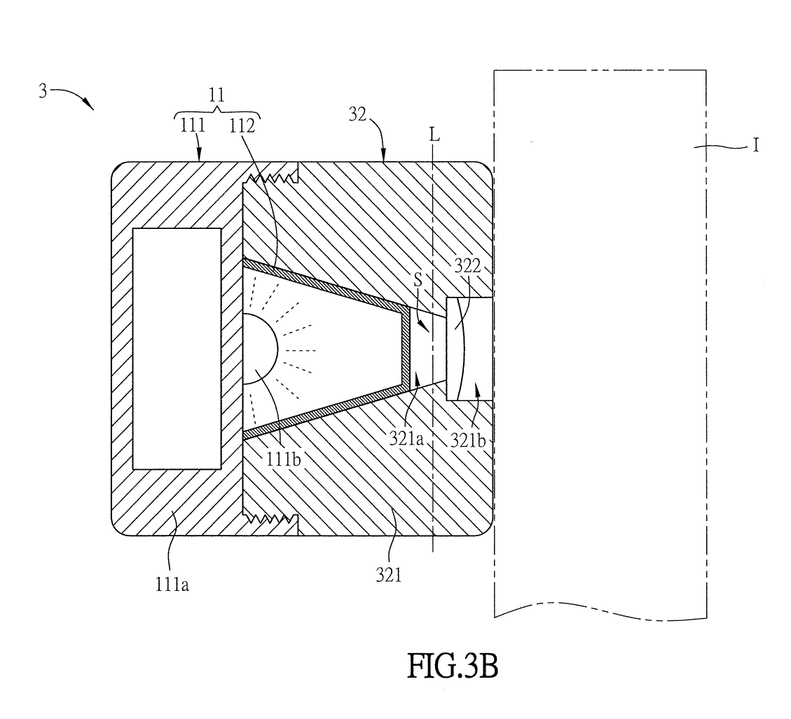

FIG. 3A is a schematic sectional exploded diagram of the sample detecting device of another embodiment of the invention taken along the line A-A, and FIG. 3B is a schematic sectional assembled diagram of the sample detecting device of another embodiment of the invention taken along the line A-A. As shown in FIGS. 3A and 3B, the composition of the sample detecting device 3 is approximately similar to the sample detecting device 1. The main difference between them is that the first cavity portion 321a of the second assembly 32 of the sample detecting device 3 communicates with the second cavity portion 321b. In this embodiment, the convex lens 322 is also disposed in the second cavity portion 321b. As shown in FIG. 3A, the side of the convex lens 322 facing the first cavity portion 321a is directly used as a bottom of the first cavity portion 321a and is a plane approximately.

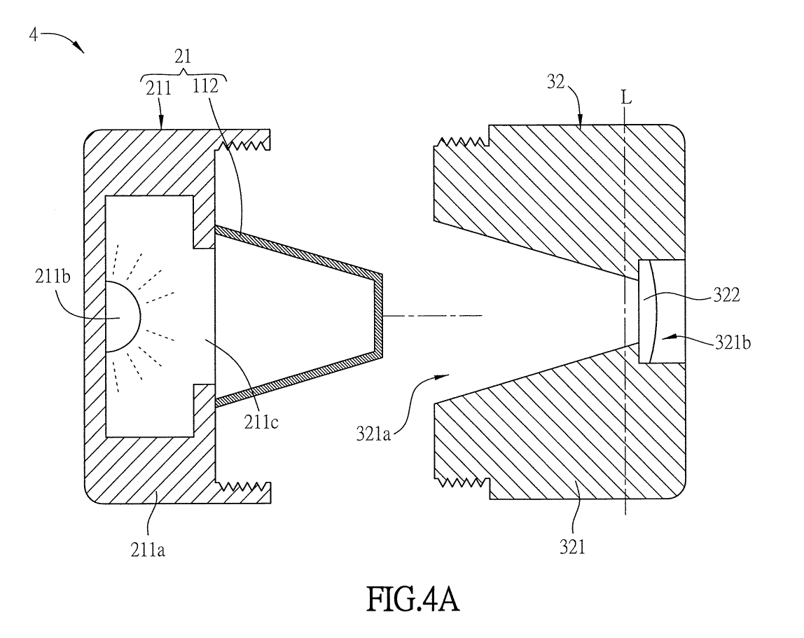

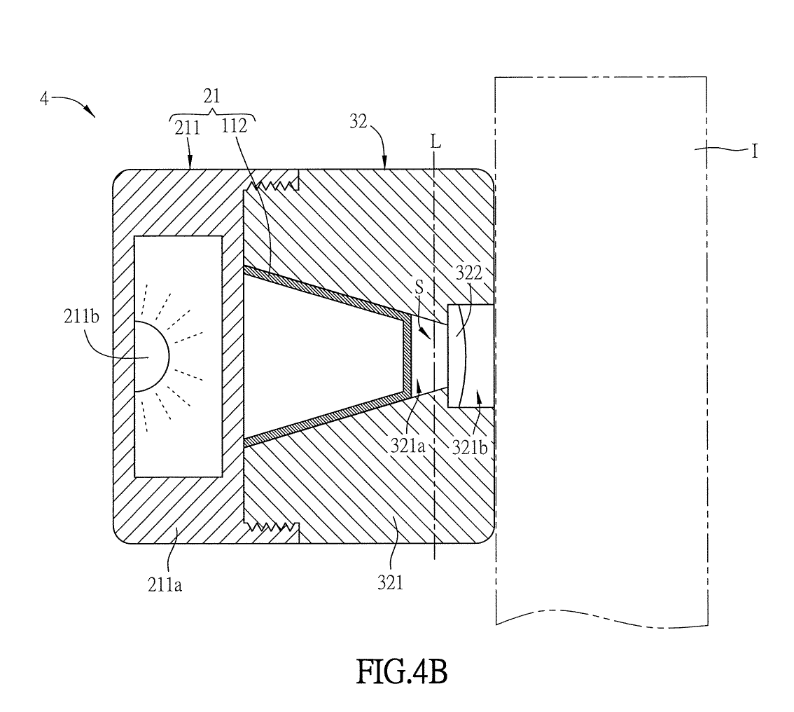

To be noted, the above embodiment is just for the illustration but not for limiting the scope of this invention. For example, as shown in FIGS. 4A and 4B, the sample detecting device 4 can be composed of the first assembly 21 and the second assembly 32. Herein, FIG. 4A is a schematic sectional diagram of the sample detecting device of another embodiment of the invention taken along the line A-A, and FIG. 4B is a schematic sectional assembled diagram of the sample detecting device of another embodiment of the invention taken along the line A-A. The detailed description of the sample detecting device 4 can refer to the above mention and therefore the related illustration is omitted here for conciseness. Moreover, the convex lens 122, 322 of the sample detecting device 1, 2, 3, 4 can be the element independently disposed in the second cavity portion 121b, 321b or can be integrally formed (ex. injection molding) with the second assembly 12, 32. According to the actual requirement, the side of the convex lens 322 of the second assembly 32 facing the first cavity portion 321a can be a plane or a curved surface, and this invention is not limited thereto.

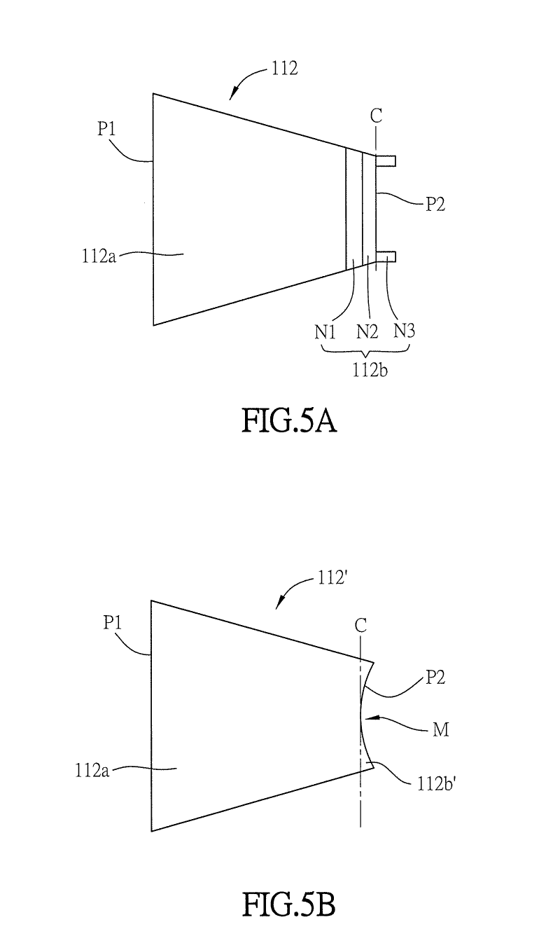

FIG. 5A is a schematic side-view of the light-permeable unit of the sample detecting device of an embodiment of the invention. In FIG. 5A, the light-permeable unit 112 includes a base portion 112a and a sample getting portion 112b. The base portion 112a includes a first connection end P1 and a second connection end P2 which is opposite to the first connection end P1. The light-permeable unit 112 is connected to the light emitting unit 111 through the first connection end P1, and the sample getting portion 112b is disposed at the second connection end P2. The sample getting portion 112b includes a soft light-permeable sub-portion N1, a hard light-permeable sub-portion N2 and a microstructure sub-portion N3. The soft light-permeable sub-portion N1 is connected with the second connection end P2. The hard light-permeable sub-portion N2 has a sample getting surface C, and the microstructure sub-portion N3 is disposed on the sample getting surface C. In this embodiment, the material of the hard light-permeable sub-portion N2 includes glass, quartz, PMMA (Polymethylmethacrylate) or PC (Polycarbonate). The material of the soft light-permeable sub-portion N1 includes silicone. The microstructure sub-portion N3 is a bar-like or granular structure, which is used to separate the first assembly 11 from the second assembly 12 to form the sample containing space S when the first assembly 11 and the second assembly 12 are assembled. To be noted, in this embodiment, the light-permeable unit 112 is characterized by the soft light-permeable sub-portion N1 disposed between the hard light-permeable sub-portion N2 and the base portion 112a. Therefore, when the first assembly 11 and the second assembly 12 are assembled, the soft light-permeable sub-portion N1 can absorb most of the stress that should cause the deformation of the hard light-permeable sub-portion N2, so that the hard light-permeable sub-portion N2 can be kept in the original shape. Thus, the volume of the sample containing space S can be fixed in size, so that the sample contained in the sample containing space S can receive a quantitative detection. The variation done by those skilled in the art in compliance with the spirit of the invention should belong to the scope of this invention.

FIG. 5B is a schematic side-view of the light-permeable unit of the sample detecting device of another embodiment of the invention. As shown in FIG. 5B, the structure of the light-permeable unit 112′ is approximately the same as the light-permeable unit 112. The main difference between them is that the sample getting portion 112b′ of the light-permeable unit 112′ includes a third cavity portion M. When the first assembly 11 and the second assembly 12 are connected, the sample containing space S is approximately formed at the third cavity portion M. After the sample getting portion 112b′ gets the sample, the sample will adhere to the third cavity portion M due to the surface adhesion. Since the surface adhesion is nearly a constant value, the sample adhering to the third cavity portion M also can be given the quantitative effect. To be noted, after reading the above mention, those skilled in the art can easily apply each of the light-permeable units 112, 112′ as mentioned in FIGS. 5A and 5B to the sample detecting device 1, 2, 3, 4, so the detailed illustration is omitted here for conciseness.

In summary, the sample detecting device of this invention can be used in cooperation with the mobile communication device. Besides, the first assembly and the second assembly can be easily assembled to form the sample containing space, so that the sample can be contained in the sample containing space and imaged. The captured microscopic image can be transmitted to the mobile communication device for the computation and thus the detection. Therefore, the threshold of the biological sample detection can be lowered down a significantly, so that the general user also can rapidly perform a simple biological sample detection at home.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A sample detecting device used in cooperation with an image capturing device, comprising:
a first assembly comprising:
a light emitting unit; and
a light-permeable unit disposed at one side of the light emitting unit; and
a second assembly matching and connecting to the first assembly to form a sample containing space, the second assembly comprising:
a body comprising a first cavity portion and a second cavity portion, wherein the light-permeable unit is disposed in the first cavity portion; and
a convex lens disposed in the second cavity portion, wherein the light emitted from the light emitting unit sequentially passes through the light-permeable unit and the convex lens and then leaves the body.

2. The sample detecting device as recited in claim 1, wherein the light emitting unit comprises a light source and a light output hole, the light source is disposed in the light emitting unit, and at least a part of the light emitted by the light source is outputted through the light output hole.

3. The sample detecting device as recited in claim 1, wherein the light emitting unit comprises a light source and a housing, the light source is disposed on a side of the housing, and the light-permeable unit is disposed on the light source.

4. The sample detecting device as recited in claim 1, wherein the first cavity portion communicates with the second cavity portion.

5. The sample detecting device as recited in claim 1, wherein the first cavity portion doesn't communicate with the second cavity portion.

6. The sample detecting device as recited in claim 1, wherein the light-permeable unit comprises:
a base portion comprising a first connection end and a second connection end opposite to the first connection end, wherein the light-permeable unit is connected with the light emitting unit through the first connection end; and
a sample getting portion disposed at the second connection end of the base portion.

7. The sample detecting device as recited in claim 6, wherein the sample getting portion comprises a third cavity portion disposed opposite to the first connection end of the base portion.

8. The sample detecting device as recited in claim 6, wherein the sample getting portion includes a soft light-permeable sub-portion, a hard light-permeable sub-portion and a microstructure sub-portion, the soft light-permeable sub-portion is connected with the second connection end of the base portion, the hard light-permeable sub-portion has a sample getting surface, and the microstructure sub-portion is disposed on the sample getting surface.

9. The sample detecting device as recited in claim 8, wherein the material of the hard light-permeable sub-portion includes glass, quartz, PMMA (Polymethylmethacrylate) or PC (Polycarbonate).

10. The sample detecting device as recited in claim 8, wherein the material of the soft light-permeable sub-portion includes silicone.

11. The sample detecting device as recited in claim 8, wherein the microstructure sub-portion is a bar-like or granular structure.

12. The sample detecting device as recited in claim 1, wherein the image capturing device is a cell phone, a tablet computer, a camera, a network camera or a driving recorder.
